(19) United States
(12) Patent Application Publication
(10) Pub. No.: US 2008/0200675 A1
(43) Pub. Date: Aug. 21, 2008

(54) Method for Production of Substituted Phenylmalonate Esters, Novel Phenylmalonate Esters and Use Thereof

(75) Inventors: Norbert Gotz, Worms (DE); Volker Maywald, Ludwigshafen (DE); Michael Keil, Freinsheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/993,947

(22) PCT Filed: Jun. 23, 2006
(86) PCT No.: PCT/EP2006/063491

§ 371 Date: Dec. 26, 2007

(30) Foreign Application Priority Data
Jun. 27, 2005 (DE) 10 2005 030 093.6

(51) Int. Cl. C07C 69/76; C07D 487/00

(52) U.S. Cl. 544/263; 560/82

(57) ABSTRACT

A process for preparing substituted phenylmalonic esters of the formula

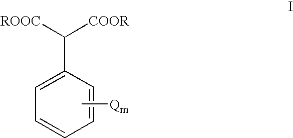

in which R is alkyl and Q is halogen, alkyl, alkoxy, haloalkyl or haloalkoxy and the index m is an integer from 1 to 5, where the groups Q can be identical or different if the index m is greater than 1, comprising steps A), B) and C):

A) reaction of compounds of the formula II,

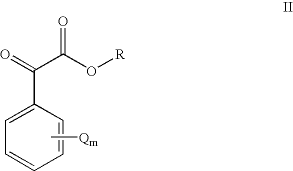

in which the variables are as defined for formula I to give compounds of the formula III,

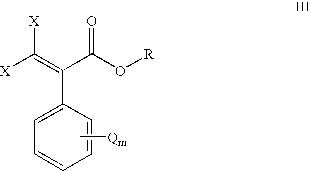

B) conversion of the compounds of the formula III into ketals of the formula IV

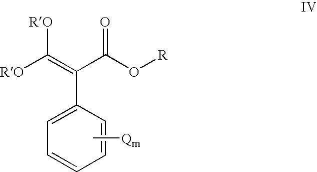

in which R′ is C₁-C₄-alkyl or benzyl,

C) hydrolysis of the compounds of the formula IV to give compounds of the formula I;

novel phenylmalonic ester derivatives, and their use as intermediates.

The present invention relates to a process for preparing substituted phenylmalonic esters of the formula I

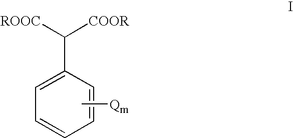

in which R is C₁-C₄-alkyl and Q is halogen, C₁-C₄-alkyl, C₁-C₄-alkoxy, C₁-C₄-haloalkyl or C₁-C₄-haloalkoxy and the index m is an integer from 1 to 5, where the groups Q can be identical or different if the index m is greater than 1, comprising steps A), B) and C):

A) reaction of compounds of the formula II,

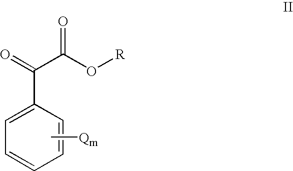

in which the variables are as defined for formula I to give compounds of the formula III,

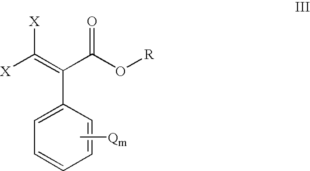

B) conversion of the compounds of the formula III into ketals of the formula IV

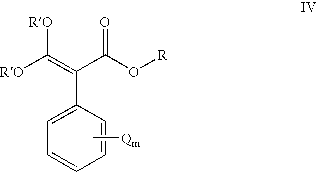

in which R′ is C₁-C₄-alkyl or benzyl,

C) hydrolysis of the compounds of the formula IV to give compounds of the formula I.

In addition, the invention relates to novel phenylmalonic ester derivatives and to their use as intermediates.

It was an object of the present invention to provide an economical process for preparing substituted phenylmalonic esters of the formula I, which process can be carried out on an industrial scale.

The prior art discloses methods for preparing phenylmalonic esters; usually, they are prepared by reacting malonic esters with aryl halides in the presence of bases [cf.: U.S. Pat. No. 6,156,925; J. Org. Chem., Vol. 67, p. 541 ff (2002); Org. Lett., Vol. 4, p. 269 ff (2002); Synth. Commun. Vol. 18, p. 291 ff (1988); GB 901 880]. Phenylmalonic esters are also accessible by condensation of phenylacetic esters with dialkyl carbonates or oxalic esters [cf. Eur. J. Med. Chem., Vol. 26, p. 599 ff (1991); J. Fluorine Chem., Vol. 59, p. 225 ff (1992); Can. J. Chem., Vol. 72, p. 2312 (1994)]. These processes have the disadvantage that, for certain phenyl substitution patterns, they give only incomplete conversions, and the end products are therefore available only in very poor yields. In the process according to U.S. Pat. No. 6,156,925, Cu-containing waste waters requiring a complicated work-up are produced. Accordingly, the known processes for preparing the compounds of the formula I are not fully suitable on an industrial scale.

We have found that the object is achieved by the process defined at the outset.

We have found that 3,3-dichloro-2-phenylacrylic esters can be converted in a simple manner, via 3,3-dialkoxy-2-phenylacrylic esters as intermediate and subsequent gentle hydrolysis, into substituted phenylmalonic esters. The process can be carried out either in two steps, with isolation of the novel 3,3-dialkoxy-2-phenylacrylic esters, or as a one-product reaction.

The process according to the invention overcomes the disadvantages of the prior art. It provides an elegant excess to substituted phenylmalonic esters, in particular those having one or more fluorine substituents in the phenyl ring. The process according to the invention is preferably suitable for preparing compounds I in which the index m is 1, 2, 3 or 4 and the group Q is fluorine, chlorine, methyl, methoxy, trifluoromethoxy, especially those in which Qₘ is 2,4,6-trifluoro.

Starting materials for the process according to the invention are phenylglyoxylic esters of the formula II which are reacted in the sense of a Wittig reaction with triphenyl-phosphine and carbon tetrahalide to give 3,3-dihalo-2-phenylacrylic esters of the formula III. The reaction is preferably carried out using carbon tetrachloride, giving the 3,3-dichloro-2-phenylacrylic esters of the formula IIIA.

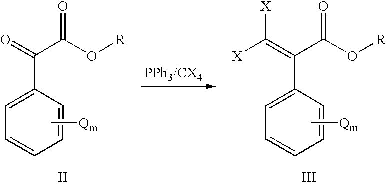

This reaction is usually carried out at temperatures of from −10° C. to 70° C., preferably from 0° C. to 20° C., in an inert organic solvent [cf. J. Am. Chem. Soc., Vol. 84, p. 1312 (1962); Tetrahedron Vol. 22, p. 2615 ff (1966); Synthetic Communications Vol. 32, p. 2821 ff (2002)].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide and dimethylacetamide, particularly preferably nitriles and halogenated hydrocarbons. It is also possible to use mixtures of the solvents mentioned.

The starting materials are generally reacted with one another in equimolar amounts. In terms of yield, it may be advantageous to employ an excess of triphenylphosphine, based on II.

The prior art discloses various methods for preparing the compounds II. They are easily accessible by coupling Grignard salts of the formula IV with oxalic esters of the formula V.

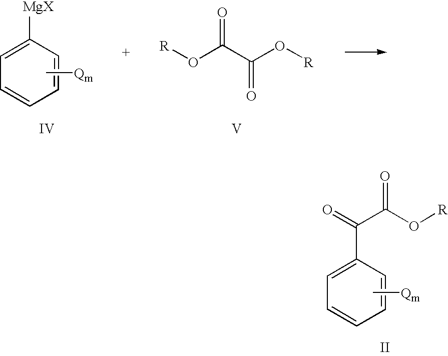

Grignard salts of the formula IV are known from the literature and can be obtained from the corresponding halobenzene derivatives, in particular bromobenzene derivatives IVA (X=Br), under generally known conditions [cf. J. Org. Chem., Vol. 52, p. 5026 ff (1987); J. Org. Chem. USSR, Vol. 24, p. 92 ff (1988)]. The Grignard reaction is usually carried out at low temperatures, preferably at temperatures of from −80° to −40° C.

In addition, the compounds II can be prepared by oxidizing the corresponding mandelic esters, by oxidizing substituted phenylacetic esters [cf. J. Chem. Soc., Chem. Commun, pp. 323-324 (1993)] or by coupling substituted phenyllithium compounds with oxalic esters [cf.: Tetrahedron Asymmetry, Vol. 8, p. 1083 ff (1997); J. Org. Chem., Vol. 68, p. 3990 ff (2003)].

Alternatively, phenylglyoxylic esters of the formula II can also be obtained by Friedel-Crafts acylation of appropriately substituted benzene derivatives of the formula VI with oxalyl halides of the formula VII [cf. J. Org. Chem., Vol. 61, p. 6407 ff (1996); J. Org. Chem., Vol. 67, p. 3585 ff (2002)]. This reaction is preferred for compounds in which at least one group is fluorine, in particular for those in which Qₘ is fluorine, chlorine or bromine.

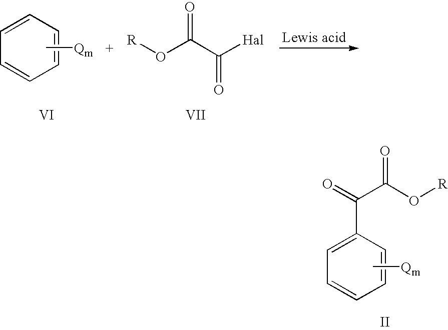

This reaction is usually carried out at temperatures of from −10° C. to 30° C., preferably from 0° C. to 15° C., undiluted or in an inert organic solvent in the presence of an acid or a catalyst.

Suitable solvents are aliphatic hydrocarbons, such as pentane, hexane, cyclohexane and petroleum ether, halogenated hydrocarbons, such as methylene chloride, 1,2-dichloroethane, chloroform and chlorobenzene, particularly preferably methylene chloride and 1,2-dichloroethane. It is also possible to use mixtures of the solvents mentioned.

Suitable for use as Lewis acids and acid catalysts are Lewis acids such as boron trifluoride, aluminum trichloride, iron(III) chloride, tin(IV) chloride, titanium(IV) chloride and zinc(II) chloride, in particular aluminum trichloride.

The acids are generally employed in catalytic amounts; however, they can also be used in equimolar amounts or in excess.

The halogen atoms of the 3,3-dihalo-2-phenylacrylic ester of the formula III are, by nucleophilic substitution, exchanged for alkoxy groups.

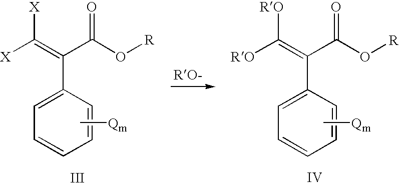

In the formula IV, R′ is C₁-C₄-alkyl, preferably methyl or ethyl. For practical reasons, the alkoxides OR′⁻ used are preferably alkali metal and/or alkaline earth metal alkoxides, in particular sodium alkoxides.

This reaction is usually carried out at temperatures of from −10° C. to 100° C., preferably from −5° C. to +50° C., in an inert organic solvent in the presence of a base.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; preference is given to hydrocarbons, ethers, and also dimethyl sulfoxide, dimethylformamide and dimethylacetamide. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. With particular preference, the base used is the alkoxide R′O⁻.

The bases are generally employed in catalytic amounts; however, they can also be used in equimolar amounts, in excess or, if appropriate, as solvent.

The starting materials are generally reacted with one another in equimolar amounts. In terms of yield, it may be advantageous to use an excess of alkoxide, based on III.

The ketals of the formula IV are hydrolyzed to give phenylmalonic esters of the formula I

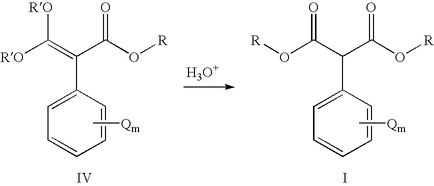

This reaction is usually carried out at temperatures of from −20° C. to +20° C., preferably from −5° C. to +15° C., in an inert organic solvent in the presence of an acid.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, preferably hydrocarbons and ether. It is also possible to use mixtures of the solvents mentioned.

Suitable for use as acids and acidic catalysts are inorganic acids, such as hydrofluoric acid, hydrochloric acid, hydrobromic acid and sulfuric acid, and also organic acids, such as formic acid, acetic acid, propionic acid, oxalic acid, toluenesulfonic acid, benzenesulfonic acid, camphorsulfonic acid, citric acid and trifluoroacetic acid. Preference is given to using diluted hydrochloric acid or acetic acid.

The acids are generally employed in excess or as solvent.

Work-up and purification of the reaction products is preferably carried out by distillation. The individual products can be identified both by HPLC and GC analysis.

The phenylmalonic esters, easily obtainable by the process according to the invention, are suitable as intermediates for preparing dyes or active compounds in the pharmaceutical or agrochemical field. In the preparation of active [1,2,4]triazolo[1,5-a]pyrimidine compounds, they are reacted with 3-amino-1,2,4-triazole to give 5,7-dihydroxy-6-phenyl[1,2,4]triazolo[1,5-a]pyrimidines [cf. EP-A 550 113, EP-A 975 634, U.S. Pat. No. 5,808,066, U.S. Pat. No. 6,117,876, WO 98/46607].

### PROCESS EXAMPLES

### Example 1

### Preparation of Ethyl (2,4,6-Trifluorophenyl)Glyoxylate

1a) At 20-25° C., 12.5 ml of a 2M isopropylmagnesium chloride solution in tetrahydrofuran (THF) were added to a mixture of 5.3 g of 2,4,6-trifluorobromobenzene in 30 ml of THF, resulting in an increase of the temperature to 54° C.

1b) A solution of 3.5 g of diethyl oxalate in 20 ml of THF was cooled to −55° C., and the Grignard solution from Ex. 1a) was added dropwise at this temperature. After 1 h at −55° C., 12.5 ml of water and then 12.5 ml of 10% strength hydrochloric acid were added at 0° C. to the reaction mixture. The aqueous phase was saturated with Na₂SO₄, the phases were separated, the aqueous phase was extracted with THF and the solvent was removed from the combined organic phases. Under a reduced pressure of 0.2 mbar, distillation of the residue gave 4.6 g of the title compound of b.p. 103° C. (80% of theory).

### Example 2

### Preparation of Ethyl 3,3-Dichloro-2-(2′,4′,6′-Trifluorophenyl)Acrylate

At 5° C., over a period of 2 h, 60 g of CCl₄ were added dropwise to a solution of 31.4 g of ethyl (2,4,6-trifluorophenyl)glyoxylate and 102 g of triphenylphosphine in 320 ml of acetonitrile. The reaction mixture was then added to 500 ml of water and subsequently extracted with methyl tert-butyl ether (MTBE). The combined organic phases were dried and the solvent was then removed. Under a reduced pressure of 0.4 mbar, distillation of the residue gave 36 g of ethyl 3,3-dichloro-2-(2′,4′,6′-trifluorophenyl)acrylate. B.p.: 68° C./0.1 mbar

### Example 3

### Preparation of Diethyl 2,4,6Trifluorophenylmalonate

At 0° C., 150 g of ethyl 3,3-dichloro-2-(2′,4′,6′-trifluorophenyl)acrylate were added drop-wise over a period of 100 min to a solution of 120 g of sodium ethoxide in 1950 ml of ethanol. After warming to 1 0C, the reaction mixture was acidified to pH 4.7 using 195 g of 10% strength acetic acid. After a further 2 h of stirring at 10° C., the mixture was extracted with methylene chloride, the organic phase was dried and the solvent was removed. Under a reduced pressure of 0.4 mbar, distillation of the residue gave 106 g (73% of theory) of the title compound. B.p.: 175.3° C./50 mbar

### Example 4

### Preparation of Ethyl 3,3-Diethoxy-2-(2′,4′,6′-Trifluorophenyl)Acrylate

At about 2° C., 10 g of the ester from Ex. 3 were added dropwise to a solution of 7.7 g of sodium ethoxide in 125 ml of ethanol. The temperature was allowed to rise to 20-25° C. and stirring was continued for about 3 h. The crystal slurry formed was taken up in 100 ml of methylene chloride and filtered, and the solvent was removed from the filtrate obtained. The residue gave 8.9 g (84% of theory) of the title compound of b.p. 112° C./0.6 mbar.

¹H-NMR δ in ppm (CDCl₃): 1.2 (t, 6H); 1.45 (t, 3H); 4-4.1 (m, 4H); 6.6 (s, 2 H).

1-9. (canceled)

10. A process for preparing a substituted phenylmalonic ester of formula I: 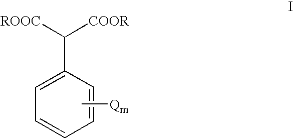
comprising the steps of:
A) converting a compound of formula II: 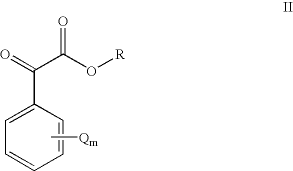
into a compound of formula III: 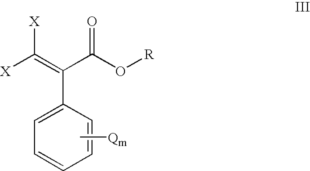
B) converting the compound of formula III into a ketal of formula IV: 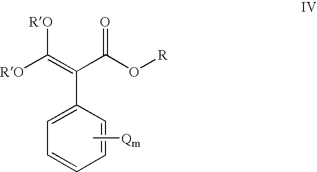
C) hydrolyzing the compound of formula IV to a compound of formula I;
wherein R is C₁-C₄-alkyl and Q is halogen, C₁-C₄-alkyl, C₁-C₄-alkoxy, C₁-C₄-haloalkyl or C₁-C₄-haloalkoxy, X is halogen, R′ is C₁-C₄-alkyl or benzyl; and m is an integer from 1 to 5.

11. The process of claim 10, wherein step A is carried out using triphenylphosphine in CCl₄.

12. The process of claim 10, wherein step B is carried out using alkali metal alkoxides.

13. The process of claim 11, wherein step B is carried out using alkali metal alkoxides.

14. The process of claim 10, wherein step C is carried out in the presence of diluted carboxylic acids or mineral acids.

15. The process of claim 11, wherein step C is carried out in the presence of diluted carboxylic acids or mineral acids.

16. The process of claim 12, wherein step C is carried out in the presence of diluted carboxylic acids or mineral acids.

17. The process of claim 10, wherein at least one group Q in formulae I, II and III is a fluorine atom.

18. The process of claim 10, wherein m is 3, Q is fluro, and Qₘ in the formulae I, II, III and IV is 2,4,6-trifluoro.

19. A process for a preparing 5,7-dihydroxy-6-phenyl[1,2,4]triazolo[1,5-a]pyrimidine comprising the steps of:
A) converting a compound of formula II: 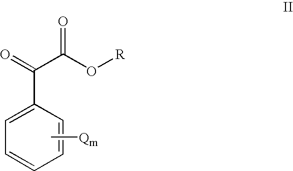
into a compound of formula III: 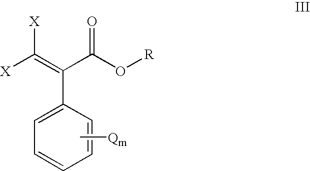
B) converting the compound of formula III into a ketal of formula IV: 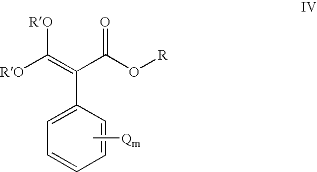
C) hydrolyzing the compound of formula IV to a compound of formula I: 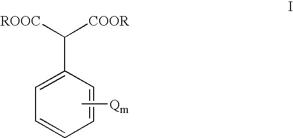
D) reacting the compound of formula I with 2-amino-1,3,5-triazole;
wherein R is C₁-C₄-alkyl and Q is halogen, C₁-C₄-alkyl, C₁-C₄-alkoxy, C₁-C₄-haloalkyl or C₁-C₄-haloalkoxy, X is halogen, R′ is C₁-C4-alkyl or benzyl; and m is an integer from 1 to 5.

20. The process of claim 19, wherein step A is carried out using triphenylphosphine in CCl₄.

21. The process of claim 19, wherein step B is carried out using alkali metal alkoxides.

22. The process of claim 20, wherein step B is carried out using alkali metal alkoxides.

23. The process of claim 19, wherein step C is carried out in the presence of diluted carboxylic acids or mineral acids.

24. The process of claim 20, wherein step C is carried out in the presence of diluted carboxylic acids or mineral acids.

25. The process of claim 21, wherein step C is carried out in the presence of diluted carboxylic acids or mineral acids.

26. The process of claim 19, wherein at least one group Q in formulae I, II and III is a fluorine atom.

27. The process of claim 19, wherein m is 3, Q is fluro, and Qₘ in the formulae I, II, III and IV is 2,4,6-trifluoro.

28. A compound of formula IVA: 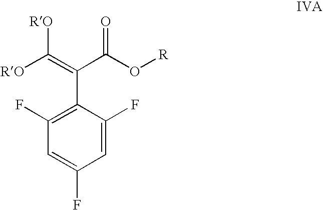
wherein R and R′ are C₁-C₄-alkyl.

29. A process for preparing a 5,7-dihydroxy-6-(2,4,6-trifluorophenyl)[1,2,4]triazolo[1,5-a]pyrimidine comprising:
reacting a compound of the formula IVA: 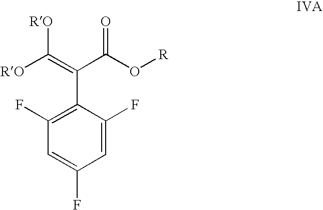
with 3-amino- 1,2,4-triazole;
wherein R and R′ are C₁-C₄-alkyl.
